(19) United States
(12) Patent Application Publication
(10) Pub. No.: US 2014/0303589 A1
(43) Pub. Date: Oct. 9, 2014

(54) FIXING AND CARRYING DEVICE FOR A DISPOSABLE ABSORBENT INCONTINENCE PAD

(71) Applicants: Rui Miguel Paz, Heidenheim (DE); Ruediger Kesselmeier, Herbrechtingen (DE); Krzysztof D. Malowaniec, Heidenheim (DE); Albena Drumeva-Eberius, Heidenheim (DE); Uwe Nagy, Heidenheim (DE); Maximilian Swerev, Augsburg (DE)

(72) Inventors: Rui Miguel Paz, Heidenheim (DE); Ruediger Kesselmeier, Herbrechtingen (DE); Krzysztof D. Malowaniec, Heidenheim (DE); Albena Drumeva-Eberius, Heidenheim (DE); Uwe Nagy, Heidenheim (DE); Maximilian Swerev, Augsburg (DE)

(73) Assignee: PAUL HARTMANN AG, Heidenheim (DE)

(21) Appl. No.: 14/112,721

(22) PCT Filed: Mar. 30, 2012
(86) PCT No.: PCT/EP12/55876

§ 371 Date: Nov. 27, 2013

(30) Foreign Application Priority Data
Apr. 20, 2011 (DE) 10-2011-007-820.7

(51) Int. Cl. A61F 13/66; A61F 13/70

(52) U.S. Cl. CPC A61F 13/665; A61F 13/70
USPC 604/395

(57) ABSTRACT

A fixing and carrying device for a disposable absorbent incontinence pad includes a girdle having a front stomach region, a rear back region and a left-hand and a right-hand side region, and girdle closure elements for detachable closure of the girdle on itself so as to form a continuous hip opening in a hip-circumferential direction, the girdle having respective flap sections extending from the back region and the stomach region in a longitudinal direction toward a crotch region of a user; and closure elements respectively provided on a body-facing side of each flap section and being constructed for interaction with complementary closure elements on a body-averted side of the incontinence pad in a detachably adhesive manner, wherein prior to use of the fixing and carrying device the closure elements are folded in onto the body-facing side of the flap section, and the flap sections are respectively folded in onto the front stomach region and rear back region.

The invention relates to a fixing and carrying device for a disposable absorbent incontinence pad, having a girdle which is detachably closable on itself by means of girdle closure elements and thus forms a continuous hip opening in the hip circumferential direction, the incontinence pad being detachably fixable to said girdle such that it can be worn in the crotch region of the user and can be removed from the girdle again and discarded after use, wherein the girdle comprises a front stomach region, a rear back region and a left-hand and a right-hand side region, and the girdle, starting from the back region and the stomach region, has in each case one flap section extending in a longitudinal direction in the direction of the crotch region of the user, said flap section having, on its side facing the body, closure elements which interact in a detachably adhesive manner with complementary closure elements on that side of the incontinence pad that faces away from the body, and wherein the closure elements of the flap section, prior to being put into use, are folded in onto that side of the flap section that faces the body and in this configuration are secured in an easily detachable manner to that side of the flap section that faces the body.

A fixing and carrying device of this type is known for example from WO 2004/069122 A1. In the case of this known fixing and carrying device, the girdle is closed on itself in the stomach region. In the left-hand and right-hand side region between the stomach region and back region, the girdle extends downward and forms in each case a flap-like attachment there, to which a comparatively widely protruding absorbent diaper unit can be secured, wherein, on that side of the absorbent diaper unit that faces the body, closure elements are provided on both sides at the front and rear for this purpose.

EP 0 700 278 B1 discloses what is known as a belt diaper having a comparatively narrow girdle which is closable on itself and to the side of which that faces away from the body, a diaper-like absorbent unit is securable.

Finally, DE 10 2009 049 463 shows a design of the generic type, from which it is already known to fold the closure elements, which are provided on the flap sections and extend in the back region and in the stomach region into the crotch region of the user, onto themselves, and thus to be able to secure closure elements which are arranged in the flap section.

This already makes it easier to put the fixing and carrying aid on.

It is now the object of the invention to further improve the ability to put the device on and also to develop a fixing and carrying device of the generic type such that, prior to being put into use, contact between the closure elements and the wearer is avoided and also, in the form in which the fixing and carrying device is provided prior to being put into use, hooking of closure elements, which are secured to the flap sections, and of girdle closure elements can be reliably prevented such that easy removal of individual products from a pack or from a stack of a plurality of products can be ensured.

This object is achieved by a fixing or carrying device of the generic type, in which the closure elements of the flap section, prior to being put into use, are folded in onto that side of the flap section that faces the body, and wherein the flap section, onto which the closure elements are folded in, is folded in onto the front stomach region and rear back region. In this case, the closure elements are brought into interaction with complementary elements of the flap section, specifically using preferably mechanically and/or adhesively acting closure elements.

By way of the flap section extending in the direction of the crotch region of the user, the girdle at the same time forms the basic configuration of a pair of briefs, which, in order to complete the shape of the briefs, only needs to be supplemented by a crotch region. In the case of the fixing and carrying device according to the invention, this crotch region is formed by the absorbent incontinence pad, wherein the rear and front flap section interact in a detachably adhesive manner with that side of the incontinence pad that faces away from the body, specifically using preferably mechanically and/or adhesively acting closure elements, after the fixing or carrying device has been put on a wearer. As a result of the fixing or the folding in of the closure elements onto the flap section and the folding in of the flap section onto the front stomach or rear back region, the in particular mechanically and/or adhesively acting closure elements are deactivated while the girdle is being put on, in particular in the case of people in need of care. They do not impede the putting on of the girdle by getting caught on the girdle or on other components such as bed sheets, patient records or the like. The fold is then opened only after the girdle has been put on, in order to fix the pad.

It is particularly advantageous in this case for the double folding in, specifically of the closure elements onto the flap section and of the flap sections onto the front or rear stomach region, to take place such that, in the correspondingly provided state, the girdle has the same extent in the longitudinal direction of the wearer, more or less along its entire length.

Preference is given to a fixing and carrying device having closure elements, folded in onto the stomach and back region, of the flap section, said fixing and carrying device having, in the stomach and back region, merely the extent in the longitudinal direction of the front stomach region or rear back region.

Preference is furthermore given to a fixing and carrying device of the above-described configuration, in which the extent of the left-hand and right-hand side region in the longitudinal direction corresponds to the extent of the front stomach region and/or of the rear back region in the longitudinal direction.

The girdle comprises in the hip circumferential direction a stomach region and a back region and two side regions which each have an extent in the hip circumferential direction and adjoin one another in the hip circumferential direction, forming a closed girdle.

In the longitudinal direction, the stomach and back region and the side regions have a proximal and a distal transverse edge which may be configured in a straight or curved manner, in particular also in an only regionally curved manner, in order to form contouring.

Finally, the distal extent of the stomach and back region and thus the distal transverse edge of the stomach and back region is defined by an imaginary line in the transverse direction at the most proximal point of the distal boundary of the girdle, for example a tangent. The most proximal point can in this case be located in the stomach, back or side region in the hip circumferential direction. In this case, the surface proximal to this distal transverse edge is a part of the stomach or back region and the surface distal to this distal transverse edge is a part of each flap section.

The longitudinal direction of the girdle on a standing user is defined in this case as the longitudinal direction and the transverse direction is in this case perpendicular thereto and coincides with the hip circumferential direction. The longitudinal direction of the incontinence pad corresponds to the direction of the longitudinal extent thereof, with the transverse direction extending perpendicularly thereto.

Particularly preferably, the incontinence pad has two longitudinal and two transverse edges, wherein, in the state secured to the fixing and carrying device, the transverse edges of the incontinence pad correspond preferably substantially to one transverse edge, in particular the transverse edge facing away from the crotch region, i.e. the proximal transverse edge, of the girdle. In other words, the proximal edge of the stomach or back region preferably coincides with a transverse edge of the incontinence pad in the put-on state. In the case of a contoured proximal transverse edge of the girdle, for example on account of a stomach cutout, the proximal edge extends parallel to the hip circumferential direction through the most distal point of this contouring. In this way, a marking can easily be provided, by way of which the optimal putting on of the girdle and also of the incontinence pad on the girdle can be achieved, and thus also positioning of the closure elements that is optimal for each particular size.

In order to improve the fit and for easy handling, it proves to be advantageous for the relevant flap section to narrow in the direction of the crotch region. It also proves to be advantageous for the closure elements of the flap section to be provided at an end of the flap section that faces the crotch. Furthermore, one or more further closure elements may be provided on the rear and/or front flap section, said further closure elements being positioned between the end, facing the crotch, of the flap section and the proximal edge of the girdle, in particular in the region of the flap sections, and serving to additionally fix the incontinence pad.

It also proves to be advantageous for the preferably mechanically and/or adhesively acting closure elements of the flap section, in particular at that end of the flap section that faces the crotch, to be provided on a strip-like material section extending transversely to the longitudinal direction, said material section being joined to the flap section. In this way, the flap section can be detachably secured over its entire two-dimensional extent to the outer side (also back sheet) of the incontinence pad in the transverse direction in an intended symmetrical manner with respect to the user. The flap section can thus be largely fold-free and, as mentioned, be spread out correctly with respect to the legs and the crotch region or with respect to a longitudinal center axis based on the user, and fixed.

Preferably, the closure elements in this position are 100 mm to 180 mm, preferably 120 mm to 170 mm, more preferably 140 mm to 160 mm away from the proximal transverse edge of the girdle in the region of the front flap section, and preferably 170 mm to 250 mm, preferably 190 mm to 240 mm, more preferably 210 mm to 230 mm away from the proximal transverse edge of the girdle in the region of the rear flap section.

Preferably, the mechanically and/or adhesively acting closure elements of the flap section have an extent in the transverse direction of 8 cm to 14 cm, preferably 10 cm to 12 cm, preferably 11 cm, and preferably an extent in the longitudinal direction of 0.5 cm to 5.0 cm, preferably 1.0 cm to 4.0 cm, preferably 1.0 cm to 3.0 cm, preferably 1.5 cm. Thus, a sufficiently large surface for the adhesive connection of the mechanically and/or adhesively acting closure elements of the flap section to the outer side of the incontinence pad is ensured for a secure fit of the product.

The outer side of the incontinence pad is in this case preferably configured such that it corresponds to the closure elements of the flap section, i.e., in the case of a mechanical closure element in the form of hooks, the outer side of the incontinence pad is formed preferably by a loop-forming component, preferably by a nonwoven, in particular the nonwoven layer of a nonwoven/film laminate, while, in the case of an adhesive closure element, the outer side of the incontinence pad is formed preferably by a film layer, to which the adhesive closure elements can adhere.

Furthermore, provision is preferably made for the flap section having closure element of the flap section folded in on it to be folded in again about a folding line extending transversely to the longitudinal direction, such that the flap section comes to lie completely on the stomach or back region, preferably on the outer side of the stomach or back region. This double folding of the flap section such that the flap section comes to lie completely on the stomach or back region, preferably both for the front and for the rear flap section, has the advantage that the fold is opened only after the girdle and the incontinence pad have been put on, in order to fix the pad, and as a result easier threading through of the girdle, which then has a uniform width substantially along the entire length of the girdle, under a lying wearer can take place and in particular adhesion of the closure elements on other items of clothing or the skin of the patient or items of laundry cannot occur.

In addition, this fold of the flap sections can be secured by fixing points. This can take place for example by means of ultrasonic connection, in particular small separate spot welds, which are fastened in the peripheral region of the overlapping layers, but also by means of adhesive connections, in particular spots of glue. Other types of adhesion are likewise covered. The opening force of this additional fixing of the flap fold is significantly below the material strength of the girdle and does not destroy the material of the girdle when the flap sections are folded open.

With regard to practical handleability, it also proves to be advantageous for the girdle to be openable and closable only in one region and for the girdle closure elements to be provided in this region at free end sections of the girdle. The girdle is thus elongate and has two end sections which are closable on one another by means of the girdle closure elements in order to be able to put the girdle around the girth of the user.

In a development of this concept of the invention, it proves to be advantageous for secondary closure elements to be provided in the other side region, in which the girdle is thus not openable and closable, by means of which secondary closure elements a circumferential length of the girdle is settable and thus at least the front flap section is positionable symmetrically with respect to the crotch of the user. Preferably, the secondary closure elements are configured such that, in the put-on state of the fixing and carrying device, they cannot be distinguished visibly and/or haptically from the girdle closure elements. However, it may also prove to be advantageous for the secondary closure elements to be distinguishable visibly and/or haptically from the girdle closure elements in order to indicate the different function of the closure elements. This may be carried out for example by different colors, shapes, textures, or in some other way. The girdle closure elements and the secondary closure elements are in this case provided advantageously on both sides of the stomach region and/or in side regions of the girdle.

When the girdle is put on, the carer guides the opened elongate girdle through under the body of the patient, with the patient lying down, at hip or back height. Then, the girdle is closed on itself by means of the girdle closure elements in order to form the closed hip opening. In this state, although the girdle has been put on, it has not been moved into its optimal fit by activating the secondary closure elements. For example, in this state, the stomach-side flap section is pulled toward the relevant side by pulling in the hip circumferential direction, said pull being exerted by the closing of the girdle closure elements. This can now be opposed by setting the optimal hip circumferential length and a suitable tension in the hip circumferential direction, in that the secondary closure elements are set optimally in a corresponding manner. By means of these secondary closure elements—as already stated—two open free ends are thus not closed on one another, but rather the circumferential length of the girdle is optionally adjusted.

The secondary closure elements can be realized in various ways. According to a preferred embodiment of the invention, it is proposed that one component of the secondary closure elements be provided on a material section joined to the girdle and the other component be provided on an outer side of the girdle itself. In such a case, the girdle as such is thus not altered, but rather it receives an additional element in the form of the material section carrying the secondary closure elements, it being possible for said material section to be attached to the material of the girdle in any desired manner, for example by sewing, adhesive bonding, sealing, ultrasonic welding or similar conventional joining methods.

It also proves to be advantageous for the girdle closure elements and/or the secondary closure elements to extend over substantially the entire extent of the girdle in the longitudinal direction, i.e. over the entire width of the girdle. In this way, when the secondary closure elements are set, a uniform pull can be exerted on the girdle over the entire width of the girdle.

It is also conceivable for the girdle closure elements and/or the secondary closure elements to taper or narrow toward their free end in order to minimize friction of the corners of these closure elements on the skin of the user.

In an alternative embodiment, it is furthermore conceivable for the girdle closure elements and/or the secondary closure elements not to extend over substantially the entire width of the girdle. In such a case, it is particularly advantageous for the transverse extent of the girdle closure elements and/or of the secondary closure elements to be greater than their longitudinal extent.

Preferably, the girdle closure elements and/or the secondary closure elements protrude above the side edge of the girdle by at least 30 mm, in particular by at least 40 mm and further particularly by at least 50 mm in the transverse direction. The extent of the girdle closure elements and/or the secondary closure elements in the longitudinal direction of the fixing and carrying device is in particular at least 25 mm, more particularly at least 35 mm and more particularly at least 45 mm. This produces a preferred ratio of transverse extent to longitudinal extent of 30 to 25, preferably of 40 to 35 and in particular of 50 to 45. In particular, it is possible to provide for the girdle closure elements and/or the secondary closure elements to be formed in a trapezoidal manner, in particular with a side ratio of 50 mm in the transverse direction to 35/30 mm in the longitudinal direction, wherein the shorter side of the trapezium forms the free side of the closure elements. Thus, a sufficiently large surface for the adhesive connection of the girdle closure elements and/or the secondary closure elements to the outer side of the girdle is ensured for a secure fit of the product.

In this case, the girdle closure elements and/or the secondary closure elements can comprise what are known as grasping or gripping sections or be connected thereto. The grasping or gripping sections are provided preferably at the free end of the girdle closure elements or secondary closure elements and have no closure means for example in the form of mechanical and/or adhesive elements. This improves the graspability of the closure means.

In a development of the invention, it is proposed to form the girdle closure elements and/or the secondary closure elements at least sectionally in an elastic manner.

In order to shorten the circumferential length of the girdle using the secondary closure elements, in this case preferably a Z-shaped fold of the girdle is brought about by closing the secondary closure elements, said fold being held by the resultant pressure of the girdle against the surface of the body of the user and/or by friction of the sections of the Z-shaped fold lying against one another.

With regard to the creation of a good fit and high wearing comfort, it proves to be advantageous for the girdle to comprise at least one elastic section, preferably an elastic section in each side region, such that the girdle is elastically stretchable in the hip circumferential direction.

It further proves to be advantageous for the girdle to be formed in a substantially non-stretchable manner in the stomach region and/or in the back region.

However, in an alternative embodiment, it may also prove to be advantageous for one or more elastic sections to be located in the stomach region and back region, preferably in the form of a wedge-shaped, trapezoidal or quadrilateral elastication. A particularly preferred alternative embodiment has two trapezoidal elastications in the back region, said elastications being in particular in the form of elastically stretchable material sections which are provided in each case in the regions of the back region which adjoin the side regions and enclose a non-elastic region between one another. In this case, the two parallel edges of the elastic material sections can be arranged in the longitudinal direction and the two further edges can coincide with a proximal transverse edge of the girdle and with an edge, directed toward the crotch region, of the back region. Depending on the width of the elastic region, provision can be made for this to extend in the longitudinal direction into the flap section, in particular the rear flap section.

With regard to the dimensioning of the girdle, it proves to be advantageous for the girdle to have, at preferably any point, an extent in the longitudinal direction of at least 5 cm, in particular of at least 6 cm, in particular of at least 7 cm, and in particular of at least 8 cm. In this case, the dimension of the girdle in the longitudinal direction, i.e. the girdle width transversely to its circumferential extent, and specifically outside the front or rear flap section, is meant. The extent of the girdle in the longitudinal direction is preferably at most 25 cm. Particularly preferably, the extent in the stomach region is less than in the back region, and the upper edge of the girdle is preferably slightly contoured in order to form a stomach cutout which improves the fit of the product, since it adapts to the wearer upon movement, and prevents the girdle from rolling/folding over in the stomach region.

With regard to the dimensioning of the flap section, it proves to be advantageous for the flap section to have an extent in the longitudinal direction over a distal transverse edge, facing the crotch, of the stomach or back region in the distal direction toward the crotch region of at least 5 cm, in particular of at least 6 cm, in particular of at least 7 cm, in particular of at least 8 cm, in particular of at least 9 cm, in particular of at least 10 cm, in particular of at most 20 cm, in particular of 10-18 cm, in particular of 10-15 cm, wherein the front and rear flap section can have the same or a different extent. The extent of the girdle in the longitudinal direction in the region of the front flap section with a contoured stomach cutout is preferably 13 cm to 19 cm, preferably 15 cm to 17 cm, in particular 16 cm, and in the region of the rear flap section is preferably 20 cm to 27 cm, preferably 22 cm to 25 cm, in particular 23.5 cm. With a contoured stomach cutout, this value was determined starting from the most distal (lowest) point of the proximal transverse edge of the stomach region.

The extent of the girdle in the transverse direction, that is to say in the hip circumferential direction, is between 75 and 175 cm, preferably between 50 and 90 cm for the size S, between 80 and 120 cm for the size M, between 100 and 140 cm for the size L, between 120 and 160 cm for the size XL, and between 140 and 190 cm for the size XXL, and follows the sizes of the clothing industry.

The width of the incontinence pad at the widest point of the product is from 200 mm to 400 mm, preferably 220 to 320 mm, and the width of the absorbent core is from 180 mm to 380 mm, preferably 200 mm to 300 mm. Thus, for example, the width of the incontinence pad MoliForm® Premium Soft Light (size 1) is 260 mm and the width of the absorbent core is 242 mm, and in the case of MoliForm® Premium Soft Extra (size 3), the width of the pad is 300 mm and the width of the absorbent core is 260 mm.

According to a preferred embodiment of the invention, the relevant flap section is formed in one piece with the stomach region and/or with the back region of the girdle. However, separately attached flap sections made from the same material as the rest of the girdle or from a different material than the rest of the girdle are likewise conceivable.

The stomach region and/or back region of the girdle is preferably formed from a nonwoven material or nonwoven composite material, in particular from a substantially non-stretchable nonwoven material or nonwoven composite material.

It further proves to be particularly advantageous for an elastically stretchable side region of the girdle to be connected non-detachably to a substantially non-stretchable stomach region and a substantially non-stretchable back region. Preferably, each side region comprises an elastically stretchable section. In an alternative embodiment, it may prove to be advantageous for parts of the back region, in particular the lateral parts of the back region which are joined to the side regions, to be formed in an elastic manner.

Furthermore, it proves to be advantageous for the front and/or rear flap section to be formed in a colored manner, wherein the sections preferably have different colors in order to serve as a visual putting-on aid.

Furthermore, it is advantageous for a marking to be applied to the front and/or rear flap section, said marking serving as a positioning aid for the incontinence pad, such that, with the incontinence pad and fixing and carrying device put on correctly, the marking on the front and/or rear flap section is brought into accordance with a visible marking on the outer layer of the incontinence pad. For example, a central recess on the edge, facing the crotch, of the front and/or rear flap section can be brought into accordance with a marking, for example in the form of a wetness indicator applied centrally in the longitudinal direction, on the outer layer of the incontinence pad. Alternatively, the width of the lower edge of the front and/or rear flap section can be selected by the manufacturer such that it can be brought into accordance with the spacing of markings on the outer layer of the incontinence pad, for example a size marking or an absorbency marking.

As already stated, it is particularly advantageous for the fixing and carrying device having closure elements, folded in onto the girdle, of the flap section to have in the region of the flap section merely the longitudinal extent in the longitudinal direction of the front stomach region or rear back region. As a result, in the folded-in state a uniform extent in the longitudinal direction is achieved along the entire length of the girdle, such that it is particularly easy to thread and pull it through under a person, and tearing does not occur in the region of the transition from the stomach or back region to the flap section.

This is even further improved if the extent of the left-hand and right-hand side region corresponds to the extent of the front stomach region or of the rear back region in the longitudinal direction. In the folded configuration according to the invention, the girdle then has the same extent in the longitudinal direction along its entire length.

Furthermore, it is preferred according to the invention for the girdle to be folded in on itself in the region of one of the side regions about a folding line extending in the longitudinal direction, such that the front stomach region and the rear back region come to lie on one another. As a result of this folding, the length of the girdle is for example approximately halved, depending on the product size, and it can be stored and packed more easily. Prior to this folding step, the girdle closure elements are opened and the girdle is in its completed folded-out configuration, as it is also when put on a wearer.

The following steps or the following folding method likewise contribute to better packability and removability from a pack.

Provision is in particular made for the closure elements of the flap section of the front stomach region and of the rear back region to come to lie over one another. As a result, kinking of the closure elements, to which the incontinence pad can be fastened, does not occur during any of the folding and folding-in steps.

Preferably, the closure elements of the flap section can be adhesively secured in the folded-in configuration in an easily detachable manner to that side of the flap section that faces the body. Alternatively, however, only folding in without adhesion can also be provided.

In order to further improve the packability and storability, the girdle can be folded in on itself about two folding-in lines extending in the longitudinal direction, said folding-in lines being provided on both sides of the front stomach region and rear back region. As a result, the transverse extent of the folded product is further reduced. Depending on the product size, for example a reduction in the already substantially halved hip circumferential length of the girdle by approximately a half to a third occurs.

Finally, provision can be made for the girdle closure element to be securable in an adhesive manner in the folded-in state to the side part not bearing the girdle closure element. As a result, the completely folded product which is ready for packing, or the packed product, is fixed to itself.

In this case, provision can be made, in the case of folding in about the folding lines arranged on both sides of the front stomach region and rear back region, for first of all that side region of the girdle that does not bear the girdle closure elements to be folded in and then for that side region of the girdle that bears the girdle closure element to be folded in. As a result, it is possible in the final step for the girdle closure element to be secured adhesively to the material of the girdle, thereby making final fixing possible.

In the case of particularly large girdles, in order to allow folding to a smaller size, provision can be made for a section of the side region which bears a part of the girdle closure element and forms a protruding region after the folding-in step about the two folding lines arranged on both sides of the front stomach region and the rear back region, to be folded in once again about a folding line extending in the longitudinal direction, such that the girdle closure element is securable to the girdle.

Where the expression longitudinal direction is used in the above text, this always relates to the longitudinal axis of a person wearing the girdle.

Finally, the invention also relates to a method for folding a fixing and carrying device for better packability and removability from a pack in particular having a plurality of girdles, wherein the method comprises the above-described folding steps.

Further features, details and advantages of the invention are provided by the accompanying patent claims and by the graphic representation and following description of a preferred embodiment of the fixing and carrying device according to the invention for a disposable absorbent incontinence pad. In the drawing;

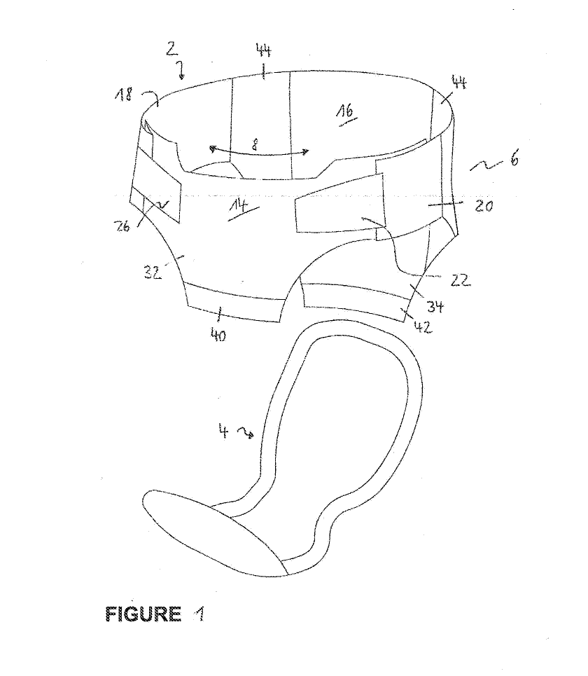

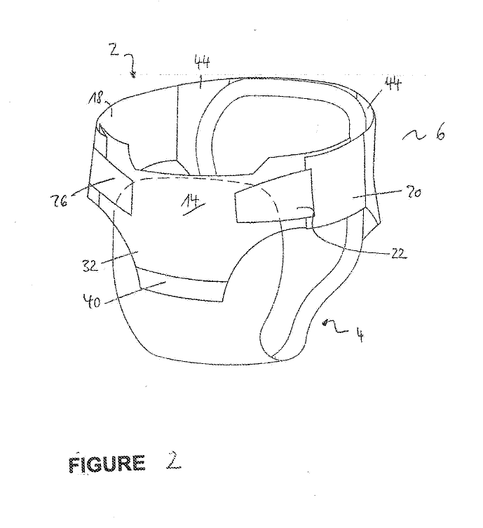

FIGS. 1 and 2 show an embodiment of the fixing and carrying device according to the invention in the put-on state together with a disposable absorbent incontinence pad;

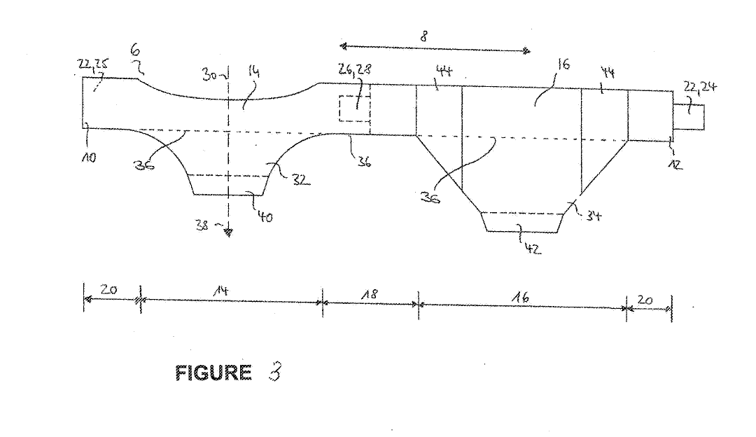

FIG. 3 shows a schematic plan view of the fixing and carrying device in the opened state spread out on a planar base, and

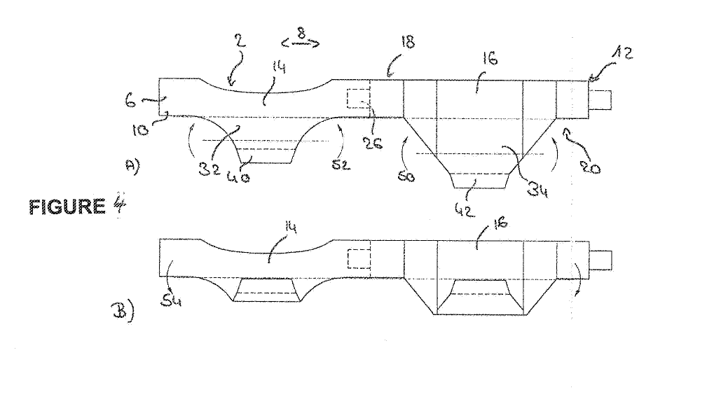

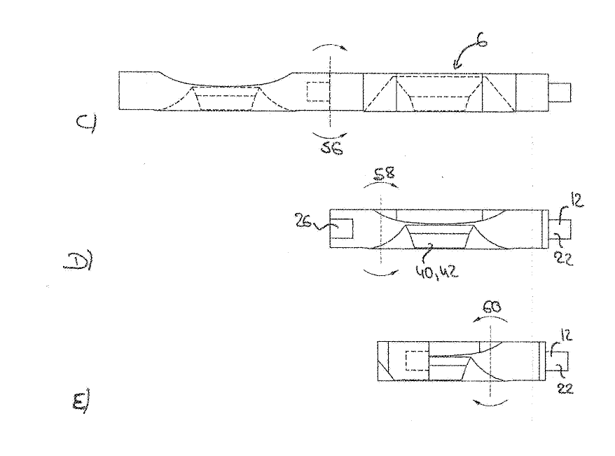

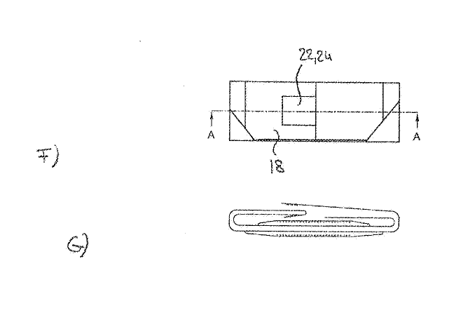

FIG. 4 shows a folding operation according to the invention.

FIGS. 1 and 2 show perspective views of a fixing and carrying device, designated overall with the reference sign 2, for an absorbent incontinence pad, indicated by the reference sign 4, specifically in the state put on a user. The fixing and carrying device 2 comprises a girdle 6 which is detachably closable on itself, said girdle 6 extending in a hip circumferential direction 8 and having two free end sections 10, 12 (see FIG. 3) which are closable on themselves to form the closed hip circumferential shape.

The fixing and carrying device 2 and its girdle 6 comprise a front stomach region 14, a rear back region 16 and a left-hand side region 18 and a right-hand side region 20.

In order to close the girdle 6, girdle closure elements 22 are provided on the free end sections 10, 12 thereof, said girdle closure elements 22 being formed in the preferred case illustrated by way of example by mechanically acting closure elements in the form of a hook/loop closure system. In this case, the hook-shaped closure component is indicated by the reference sign 24 in the figures, said closure component projecting beyond the side edge 12 of the girdle and interacting in a detachably adhesive manner with a loop-forming component 25 in the form of the outer surface of the stomach region 14. For example, the outer side of the stomach region 14 has a nonwoven material which forms the loop-like component 25 of the closure system.

Furthermore, the girdle 6, which is openable only at its end sections 10, 12, i.e. only at one point, has secondary closure elements 26, by means of which the circumferential length of the girdle 6 is settable. The secondary closure elements 26 are provided laterally on the stomach region 14 and on the left-hand side region 14 such that, in the put-on state of the fixing and carrying device (see FIG. 1), they give the same optical impression as the girdle closure elements 22 with regard to formation and arrangement, although their function is different. Specifically, by means of the secondary closure elements 26, no end sections of the girdle 6 which are openable and seperable from one another are connected together, but rather the secondary closure elements 26 serve merely to adapt the intended fit of the girdle 6 to the wearing situation. For this purpose, the secondary closure elements 26 comprise a material section 28 attached to the outer side of the girdle 6, the secondary closure elements 26 themselves being provided thereon. The secondary closure elements 26 are again preferably mechanically acting closure elements, in particular hook/loop materials. In the preferred case illustrated by way of example, the secondary closure elements 26 are formed such that a hook-forming component of the closure system is provided on the material section 28, said hook-forming component interacting with a loop-forming outer side of the stomach region 14, as was described previously in the case of the girdle closure elements 22.

As a further essential component, the girdle 6 comprises, starting from the stomach region 14 and from the back region 16, in each case a front and rear flap section 32, 34 formed symmetrically with respect to a longitudinal center axis 30. In the case preferably illustrated by way of example, the flap sections 32, 34 are formed in one piece with the girdle 6; they extend from a lower distal transverse edge 36, facing the crotch, of the girdle along the longitudinal center axis 30 in the direction 38 of the crotch region. They can narrow in the direction of the crotch region and be formed so as to be contoured in laterally arcuate manner. At their lower end region facing the crotch, the flap sections 32, 34 comprise mechanically acting closure elements 40, 42, specifically on the side facing the body. These mechanically acting closure elements 40, 42 interact in a detachably adhesive manner with complementary mechanically acting closure elements on that side of the incontinence pad 4 that faces away from the body, in order to secure the incontinence pad 4 in a detachable but captive manner on the fixing and carrying device 2. Preferably, the closure elements 40, 42 are the hook-forming component of a mechanically acting closure system, said hook-forming component interacting with the loop-forming component on that side of the incontinence pad 4 that faces away from the body. The loop-forming component can in this case be formed advantageously by a nonwoven coating, preferably the nonwoven layer of a nonwoven/film laminate, of the incontinence pad 4.

The girdle 6 is preferably formed from nonwoven materials or nonwoven composite materials. In the case which is illustrated by way of example but is preferred, the stomach region 14 and the two side regions 18, 20 are formed from a substantially non-stretchable nonwoven material or nonwoven composite material, the outer side of which serves as the loop-forming component for the girdle closure elements 22 or the secondary closure elements 26. The back region 16 is formed at least partially in an elastically stretchable manner, in that it has a pair of elastically stretchable material sections 44 which are connected in an intended non-detachable manner to the non-stretchable material of the side regions 18, 20 and enclose an elastically non-stretchable region of the back region between one another.

The fixing and carrying device 2 together with the absorbent incontinence pad 4 is put on as follows: first of all, in the case of a bedridden patient in need of care, a carer guides the girdle 6 through under the body of the patient at hip height. In the process, the girdle 6 is positioned such that the back region 16 comes to lie underneath the buttocks and the stomach region 14 comes to rest approximately centrally at the front. Then, the free end sections 10, 12 of the girdle 6 are positioned one above the other and are closed on one another by means of the girdle closure elements 22. By subsequently positioning and closing the material section 28 and the secondary closure elements 26, the length and tension of the girdle 6 in the circumferential direction 8 is optimized, such that a uniform pull on the stomach region 14 to the left and right is exerted and the stomach region 14 and also the back region 16 come to rest preferably symmetrically with respect to the crotch region of the patient and as far as possible in a fold-free manner. In this case, wearing comfort is also an important factor. Then, the front and rear flap section 32, 34 is connected in a detachably adhesive manner to the incontinence pad 4, previously or only now positioned in the crotch region of the patient, using the mechanically acting closure elements 40, 42. Here too, care should be taken to ensure that the front and rear flap sections 32, 34 are fixed to the incontinence pad 4 with a uniform and moderate pulling or holding force being exerted, in order that the incontinence pad 4 remains in its intended symmetrical arrangement in the crotch region of the patient.

As far as the order of the above-outlined steps for putting on the sanitary article or incontinence system is concerned, the carer is comparatively free. For example, in certain care situations, it may prove to be advantageous for the incontinence pad first of all to be pre-positioned in the crotch region of the patient and only then for the girdle 6 to be closed on itself. It may also prove to be expedient for the secondary closure elements 26 to be used only after the flap sections 32, 34 have been connected to the incontinence pad 4, in order to predefine the optimal tension in the hip circumferential direction 8.

FIG. 4 now shows how an article according to the invention can be folded and folded together for easier packability and removability from a pack, in particular also when a plurality of fixing and carrying devices 2 are arranged in a pack. The fixing and carrying device 2 is in this case shown in illustration A) in plan view onto the inner side with its two free end sections in a folded-open state, said end sections being closable on themselves to form the closed hip circumferential form. In the front stomach region 14 and in the rear back region 16, a flap section 32 and 34 is in each case attached to the stomach region 14 and back region 16, a closure element 40 and 42, respectively, being arranged on the lower edge region, facing the crotch, of said flap section 32 and 34. In this case, only the lower part of the flap sections 32 and 34, which bear the preferably one-piece closure elements 40 and 42 extending preferably over the entire width of the flap sections, is folded over upward about a folding line 50, 52 onto the upper region of the flap sections 32 and 34, such that the closure elements 40 and 42 come to lie in an adhesive manner on the material of the flap sections, in that the closure elements 40 and 42, which are directed toward a wearer in the wearing state, interact in an adhesive manner, in particular a mechanically adhesive manner, with the material, directed toward a user, of the flap section 32 and 34, respectively. In a next folding step, which is shown in illustration B), the upper flap region of the flap sections 32 and 34 is then folded in about a folding-in line designated 54, such that the flap regions, onto which closure elements have been folded in, come to lie on the rear back region 16 or the front stomach region 14. After this folding and folding-in step, the girdle 6 has, substantially along its entire length, the same extent in the longitudinal direction, which is indicated by the reference sign L in illustration C).

A correspondingly folded-in girdle 6 is shown in illustration C). The girdle 6 is then folded in onto itself about a folding-in line 56 extending in the longitudinal direction and thus extending at a right angle to the folding-in lines 50 to 54, such that the closure elements 40 and 42 come to lie over one another in the folded-in state. In this case, the girdle 6 is substantially halved with regard to its longitudinal extent.

After this folding step, the free end section 12, which bears the girdle closure element 22 having the hook-forming component 24, projects beyond the free end section 10 in the transverse direction. The folding line is in this case selected such that a secondary closure element 26 is not kinked by the folding line. There now follows a further folding step, shown in illustration D), about a folding line 58 extending in the longitudinal direction, wherein the folding line 58 does not come to overlap the closure elements 40 and 42 such that the side part 18, which has the secondary closure element 26, is folded in about this line such that the secondary closure element 26 comes to lie in the region of the back and stomach region 16, 14. In a following folding step, which is shown in illustration E), the second side part 20 is likewise folded in about a further folding line 60 extending in the longitudinal direction, wherein then the free end section 12 having the girdle closure element 22 comes to lie such that it can be secured adhesively to the outer material of the side part 18 by way of its in particular mechanical and/or adhesive closure elements, in particular by way of the hook-forming component 24. The final folding can be gathered in this case from illustration F).

Illustration G) shows the folded girdle as per illustration F) in a sectional illustration along the line A-A.

If the fixing or carrying device is one having a large hip circumferential width, provision can furthermore be made for the side part 20, in particular the free end section 12, which bears the hook-forming component 24 of the girdle closure element 22, to be wrapped once around the finally folded fixing and carrying device 2 and only then for the hook-forming component 24 of the girdle closure element 22 to be secured to the outer material of the girdle 6.

In this way, all of the closure elements 22, 26, 40, 42 can be fixed as easily as possible in the packed state, such that it is possible to prevent the closure elements, i.e. the secondary closure elements, the girdle closure elements and the closure elements for securing the incontinence pad, from interacting adhesively, during packing and/or removal from a pack, with a further fixing and carrying device and thereby preventing easy removability.

Furthermore, when the fixing and carrying device is put on a patient, as a result of the folding in of the flap sections, accidental adhesion of the closure elements 40 and 42 to items of clothing or other materials does not occur.

1.-16. (canceled)

17. A fixing and carrying device for a disposable absorbent incontinence pad, said fixing and carrying device comprising:
a girdle comprising a front stomach region, a rear back region and a left-hand and a right-hand side region, and girdle closure elements for detachable closure of the girdle on itself so as to form a continuous hip opening in a hip-circumferential direction, said girdle having respective flap sections extending from the back region and the stomach region in a longitudinal direction toward a crotch region of a user; and
closure elements respectively provided on a body-facing side of each said flap sections and being constructed for interaction with complementary closure elements on a body-averted side of the incontinence pad in a detachably adhesive manner, thereby enabling detachable attachment of the incontinence pad to the girdle,
wherein prior to use of the fixing and carrying device the fixing and carrying device has a folded-in configuration in which the closure elements are folded in onto the body-facing side of the flap section, and the flap sections are respectively folded in onto the front stomach region and rear back region.

18. The fixing and carrying device of claim 17, wherein an extent of the fixing and carrying device in the stomach and back region in the longitudinal direction corresponds to an extent of the front stomach region or rear back region in the longitudinal direction.

19. The fixing and carrying device of claim 18, wherein an extent of the left-hand and right-hand side region in the longitudinal direction corresponds to the extent of the front stomach region and/or of the rear back region in the longitudinal direction.

20. The fixing and carrying device of claim 17, wherein the girdle is folded in on itself in a region of one of the side regions about a folding line extending in the longitudinal direction, such that the front stomach region and the rear back region come to lie on one another.

21. The fixing and carrying device of claim 20, wherein the closure elements of the flap sections of the front stomach region and of the rear back region come to lie over one another.

22. The fixing and carrying device of claim 20, wherein the girdle is foldable in on itself about two folding lines extending in the longitudinal direction, said folding-in lines being provided on both sides of the front stomach region and rear back region and outside the closure elements of the flap sections.

23. The fixing and carrying device of claim 22, wherein the girdle closure elements are arranged on one of the side regions, and wherein the side regions are folded in about the two folding lines by first folding in the one of the side regions and then folding in the other one the side regions.

24. The fixing and carrying device of claim 23, wherein when the side regions are folded in, the girdle closure element is securable in an adhesive manner on the other one of the side regions.

25. The fixing and carrying device of claim 23, wherein a free end section of the one of the side regions bears a part of the girdle closure element, is folded in about the girdle about a folding line extending in the longitudinal direction, such that the girdle closure element is securable to the girdle.

26. The fixing and carrying device of claim 17, wherein in the folded-in configuration the closure elements of the flap section are adhesively secured to the body-facing side of the flap section in an easily detachable manner.

27. The fixing and carrying device of claim 17, wherein in the folded-in configuration the flap sections are detachably fixed on the front stomach region and rear back region in a detachable manner, in particular by means of ultrasonic welded connections or adhesive connections.

28. The fixing and carrying device of claim 17, further comprising secondary closure elements for adjusting a circumferential length of the girdle, thereby enabling positioning of at least the flap section extending from the stomach region symmetrically in relation to the crotch of the user, said secondary closure elements being provided in one of the side regions of the girdle, opposite the girdle closure elements.

29. The fixing and carrying device of claim 17, wherein the girdle is openable and closable only in one region and the girdle closure elements are provided in the one region on free end sections of the girdle, and wherein the girdle closure elements are provided in one or both of the left and right side regions of the girdle.

30. The fixing and carrying device of claim 28, wherein the girdle closure elements and/or the secondary closure elements and/or the closure elements of the flap sections are mechanical and/or adhesive closure elements.

31. The fixing and carrying device of claim 17, wherein the girdle is openable and closable only in one region and the girdle closure elements are provided on a free end section of the one region.

32. The fixing and carrying device of claim 17, wherein the closure elements of the flap section are provided on a strip-like material section extending transversely to the longitudinal direction, said material section being joined to the flap section.
